(19) United States
(12) Patent Application Publication
(10) Pub. No.: US 2018/0256089 A1
(43) Pub. Date: Sep. 13, 2018

(54) ASSESSMENT SYSTEM AND METHOD FOR DETERMINING AT LEAST ONE OF MACRO-TOPOLOGY, MILLI-TOPOLOGY, MICRO-TOPOLOGY AND NANO-TOPOLOGY

(71) Applicant: University of Oulu, University of Oulu (FI)

(72) Inventors: Heikki Nieminen, Helsinki (FI); Tuomo Ylitalo, Helsinki (FI); Simo Saarakkala, Oulu (FI); Edward Haeggström, Helsinki (FI)

(73) Assignee: University of Oulu, University of Oulu (FI)

(21) Appl. No.: 15/977,832

(22) Filed: May 11, 2018

Related U.S. Application Data
(63) Continuation of application No. PCT/FI2016/050797, filed on Nov. 11, 2016.

(30) Foreign Application Priority Data
Nov. 13, 2015 (FI) 20155841

(51) Int. Cl. A61B 5/00; G06T 7/00; G06T 7/62; G06T 7/11; G06T 7/187

(52) U.S. Cl. CPC A61B 5/4514; G06T 7/0014; G06T 2207/30024; G06T 7/11; G06T 7/187; G06T 7/62

(57) ABSTRACT

An assessment method is disclosed to determine at least one of macro-topology, milli-topology, micro-topology and nano-topology of at least one interface of at least two media using topology of the interface. Topology information of the interface is processed by performing segmentation of volume information of the obtained information from background information of the obtained information. Reference surface information is generated and information on voids is obtained and analyzed to provide multivalued surface shape information. Quantitative mapping of the information on voids is performed using the multivalued surface shape information for determining at least one of macro-topology, milli-topology, micro-topology and nano-topology of the interface.

### RELATED APPLICATION

This application claims priority as a continuation application under 35 U.S.C. § 120 to PCT/FI2016/050797, which was filed as an International Application on Nov. 11, 2016 designating the U.S., and which claims priority to Finnish Application 20155841 filed in Finland on Nov. 13, 2015. The entire contents of these applications are hereby incorporated by reference in their entireties.

### FIELD

The present disclosure relates to analyzing properties of matter. For example, the present disclosure relates to a method and a computer program for determining at least one of macro-topology, milli-topology, micro-topology, and nano-topology of the top surface of articular cartilage (TSAC) of which parts can be embedded inside articular cartilage (AC). A corresponding imaging method, imaging system, and components thereof are also disclosed. Applications for characterizing complex multivalued surface topologies extend to, for example, characterizing AC degeneration, nano-particles, cellulose fibers, bio-mimetic surfaces such as non-wetting tissue as well as macro-topologies such as land erosion, seabed, and asteroids. In particular AC degeneration stages can be classified based on the proposed approach.

### BACKGROUND INFORMATION

Assessment of at least one of surface milli-topology, micro-topology, and nano-topology of TSAC can be important both for research and clinical work related to osteoarthritis (OA) since the surface topology of TSAC is complex and strongly depends on the degenerative stage of the AC. Such topological assessment can be relevant to characterizing other diseases as well, e.g. osteoporosis.

Current assessment techniques of OA are mostly 2D (e.g. histology, i.e. tissue sectioning, staining and imaging by optical microscopy), they are subjective and they do not provide confidence limits necessary for probability-of-correct-classification analysis. In techniques such as histology the pathological state of AC (articular cartilage) is evaluated by visual inspection. This subjects the approach to intra-user and inter-user variability. Quantitative, automatic, user-independent techniques to compute pathology-related parameters (e.g., average roughness) can overcome the problems related to subjective reader-induced bias. However, the classical surface roughness measures fail to characterize for instance multivariate 3D surface features such as complex fissures that are known to be clinically relevant [Pritzker et al. Osteoarthritis Cartilage. 2006 January; 14(1):13-29].

There are articles, patents, and standards describing surface characterization, both methods and algorithms [Maerz et al. Osteoarthritis Cartilage. 2015 Oct. 5. pii: S1063-4584(15)01320-5; Brill et al. Biomed Opt Express. 2015 Jun. 8; 6(7):2398-411; Liukkonen et al. Ultrasound Med Biol. 2013 August; 39(8):1460-8; WO 2009052562 A1; U.S. Pat. No. 8,706,188 B2; US 20150153167 A1; US 20150059027 A1; U.S. Pat. No. 6,739,446 B2; ISO 25178-2:2012]. Both qualitative and quantitative measures are used, but are only valid for characterizing an unambiguous surface topology. Briefly, there are no standards for multivalued surfaces and even current standards focus on flat surfaces and curved surfaces to a lesser degree [ISO 25178-2:2012]. Few of the aforementioned methods can map surface roughness with high spatial resolution, but rather provide a few global parameters that describe the entire surface rather than local topology.

The article [Moussavi-Harami et al. J Orthop Res. 2009; 27(4):522-8] describes characterization of AC based on automation of Mankin scores (pathological AC degeneration scoring based on optical images from stained AC-bone sections). Other articles describe characterization of TSAC integrity based on known or standard engineering roughness parameters [Maerz et al. Osteoarthritis Cartilage. 2015 Oct. 5. pii: S1063-4584(15)01320-5; Brill et al. Biomed Opt Express. 2015 Jun. 8; 6(7):2398-411; Liukkonen et al. Ultrasound Med Biol. 2013 August; 39(8):1460-8]. All of these methods determine TSAC as an unambiguous surface; however, TSAC is ambiguous and multivalued. Therefore, the existing selection of standard roughness parameters for evaluation of AC surface integrity is not sufficiently descriptive to capture the complexity of TSAC typical to OA.

### SUMMARY

A material assessment system is disclosed for determining at least one of macro-topology, milli-topology, micro-topology and nano-topology of at least one interface of at least two media, the system comprising: means for obtaining information on a topology of at least one interface of at least two media; means for importing the obtained information from the obtaining means; a data-analysing unit for receiving the obtained information, the data-analysing unit having algorithmic means for processing the obtained information on the topology of the at least one interface of at least two media by performing segmentation, in which volume information of the obtained information is segmented from background information of the obtained information; means for generating reference surface information; means for obtaining information on voids; means for analyzing the information on voids by applying a region growing algorithm to provide complex multivalued surface shape information; means for performing quantitative mapping of the information on voids based on the multivalued surface shape information; and wherein the data-analysis unit is configured for determining at least one of macro-topology, milli-topology, micro-topology and nano-topology of at least one interface of at least two media, said data-analysing unit being configured for processing the obtained information on the topology of the at least one interface of at least two media by determining roughness topology of the multivalued surface of said at least one interface based on a mathematical equation.

A material assessment method is also disclosed for determining at least one of macro-topology, milli-topology, micro-topology and nano-topology of at least one interface of at least two media, wherein the method comprises: obtaining information on the topology of at least one interface of at least two media; importing the obtained information to data-analysis, wherein the obtained information on the topology of the at least one interface of at least two media is processed by performing segmentation, in which volume information of the obtained information is segmented from background information of the obtained information; generating reference surface information and obtaining information on voids; analyzing the information on voids by applying a region growing algorithm to provide complex multivalued surface shape information; quantitatively mapping the information on voids based on the multivalued surface shape information; and determining at least one of macro-topology, milli-topology, micro-topology and nano-topology of at least one interface of at least two media by is processing the obtained information on the topology of the at least one interface of at least two media by determining roughness topology of the multivalued surface of said at least one interface based on a mathematical equation.

### BRIEF DESCRIPTION OF THE DRAWINGS

Other features and advantages of embodiments of the present disclosure will become more apparent from the following detailed description when read in combination with the drawings, wherein like elements are represented by like reference numerals, and wherein:

FIG. 1 presents a block diagram of a method and computer program according to an exemplary embodiment;

FIG. 2 presents exemplary steps for identifying a reference surface and a void between TSAC and reference surface;

FIG. 3 presents a graphical presentation of geometrical aspects for determining quantitative parameters related to TSAC topology; and

FIG. 4 presents exemplary quantitative maps (Maximum depth of the voids, Tortuosity-like parameter and Depth-wise integral) determined for AC from a patient with OA (osteoarthritis).

### DETAILED DESCRIPTION

The present method and assessment system can provide significant improvement for determination of at least one of macro-topology, milli-topology, micro-topology and nano-topology of at least one interface of at least two media on the basis multivalued surface shape information. This is achieved by a material assessment system for determining at least one of macro-topology, millitopology, microtopology and nanotopology of at least one interface of at least two media. The system can include means for obtaining information on the topology of the of at least one interface of at least two media. The assessment system can include a processing unit for processing the obtained information on the topology of the of at least one interface of at least two media by performing segmentation, in which volume information of the obtained information is segmented from background information of the obtained information, by generating reference surface information, by obtaining information on voids, by analyzing the information on voids to provide multivalued surface shape information, and by performing quantitative mapping of the information on voids on the basis of the multivalued surface shape information for determining at least one of macro-topology, milli-topology, micro-topology and nano-topology of at least one interface of at least two media.

A focus of the disclosure is also a material assessment method, which method determines at least one of macro-topology, milli-topology, micro-topology and nano-topology of at least one interface of at least two media, and obtains information on the topology of the at least one interface of at least two media. The method processes the obtained information on the topology of the at least one interface of at least two media by performing segmentation, in which volume information of the obtained information is segmented from background information of the obtained information, reference surface information is generated, information is obtained on voids, the information on voids is analyzed to provide multivalued surface shape information, and quantitative mapping of the information on voids is performed on the basis of the multivalued surface shape information for determining at least one of macro-topology, milli-topology, micro-topology and nano-topology of at least one interface of at least two media.

The disclosure is based on segmentation, in which volume information of the obtained information is segmented from background information of the obtained information, on generation of reference surface information, and on analysis of the information on voids to provide multivalued surface shape information. The disclosure can also be based on quantitative mapping of the information on voids on the basis of the multivalued surface shape information for determining at least one of macro-topology, milli-topology, micro-topology and nano-topology of at least one interface of at least two media.

A benefit of the disclosed embodiments is significant improvement for determination of at least one of macro-topology, milli-topology, micro-topology and nano-topology of at least one interface of at least two media on the basis multivalued surface shape information.

A method and computer program are disclosed to automatically extract objective and robust measures of complex TSAC (Top Surface of Articular Cartilage) topology on nanometer to millimeter scale, which are pathologically and clinically relevant to diagnosis and treatment of OA (osteoarthritis). The method can include sample volume segmentation, reference surface generation, void extraction, and void analysis. A void refers to the volume “trapped” between the reference surface and TSAC. Exemplary embodiments allow objective user independent OA diagnosis and therapy monitoring (main benefit).

There is a clear need for a method that can automatically and, optionally, semi-automatically or manually extract objective and robust 3D measures that are based on or derived from pathologically or clinically relevant features for diagnosis and treatment of OA. Technically, it should provide a nondestructive, user-independent quantification of complex multivalued topology in TSAC. It should also allow producing images that can be compared to existing gold standards, e.g. histology.

An exemplary objective of the disclosure is to provide an automatic and quantitative user independent method for determining clinically relevant information, including at least one of surface macro-topology, milli-topology, micro-topology, and nano-topology of TSAC with complex structure. An exemplary aim is to provide a computer program and a system for automatic and quantitative user independent determination of clinically relevant milli-/micro-/nano-topology of TSAC. The presented methodology can differentiate the early AC degeneration stages in Pritzker et al., preferably, for example, grades 0-3, which are clinically most important [Pritzker et al. Osteoarthritis Cartilage. 2006 January; 14(1):13-29].

The following definitions are given to some main terms which are related to the present disclosure: The term “segmentation” covers, for example, algorithms intended to extract embedded volumes of interest within a volume by recognizing relevant boundaries. The process can be iterative. The term “automatic” covers the situation where no or minimum operator interference is required. It also covers the situation where the operator either carries out one step or oversees the automatic algorithm. The term “multivalued” includes situations where there are overhangs in the surface structure (along the z-axis the surface is multivalued, that is it has many points; i.e., it is folded). The term “robust” includes void characterization that does not change much depending on imaging parameters and algorithm parameters and operator. For instance variation in image intensity by less than 10% alters the depth of cleft estimate by less than 10%. The term “clinically relevant” includes an output of a disclosed method affects clinical assessment and or diagnosis and or treatment. The term “clinically founded” includes a parameter (biomarker) chosen based on features that are generally accepted as being clinically relevant for staging or prognosis; e.g., from the extended OARSI grading scheme. The term “confidence limit” indicates uncertainty and bias in an estimate based on statistical fluctuations (noise) in input data and or algorithmic model or parameter change and or imaging parameters or calibration. The term “standard” includes agreed on classification of results used to unify a method across the globe.

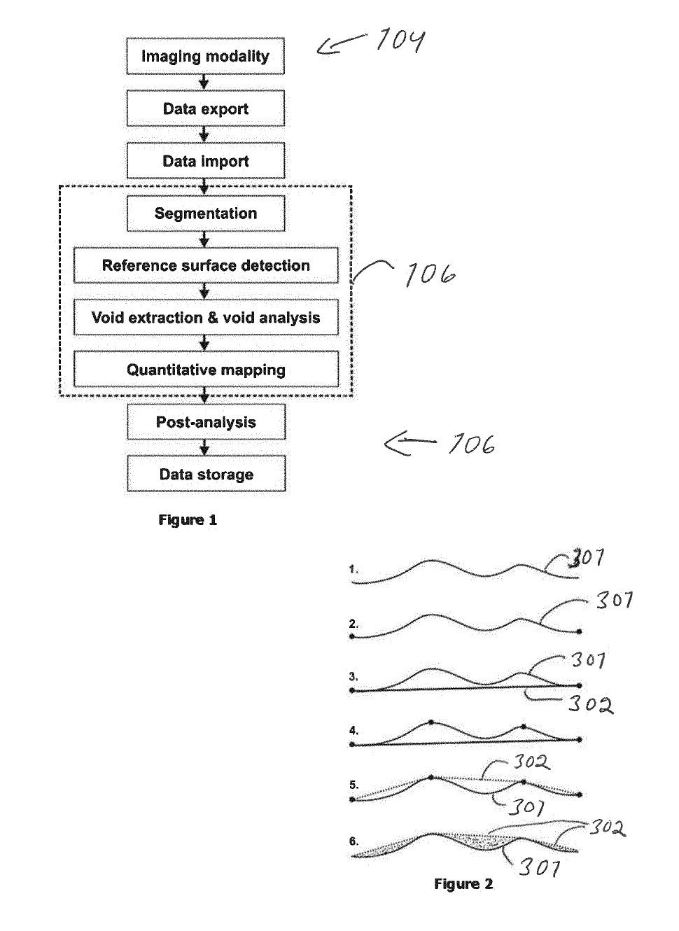

FIG. 1 presents an overview of exemplary basic components and analysis steps of a present characterization system according to an exemplary embodiment disclosed herein. The system (FIG. 1) includes 1. an imaging modality unit, e.g. μCT, with data export module, 2. data import module that can handle the 3D image output of the imaging unit, 3. data-analysis unit & program (segmentation, reference surface detection, void extraction & void analysis, quantitative mapping), 4. post-analysis unit & program and 5. means for data storage (e.g., digital memory). In particular, the data-analysis unit and the program can determine the milli/micro/nano-topology of TSAC. In addition to the components described above the computer program includes means to ensure the integrity of input and output data as well as means to ensure that characterization carried out across different samples and across different measurement sessions are commensurate (e.g., dedicated software modules). In addition, to the components described above, the computer program can include means for calculating confidence limits for the presented parameters as well as calculating probability of correct classification. The method and computer program can be implemented as software, firmware and/or hardware modules on presently known or prospective computing devices such as microcontrollers, FPGA architechtures, rasbery-pi and singleboard chip computers, laptop computers, desktop computers, supercomputers, distributed cloud computing systems, ASIC platforms.

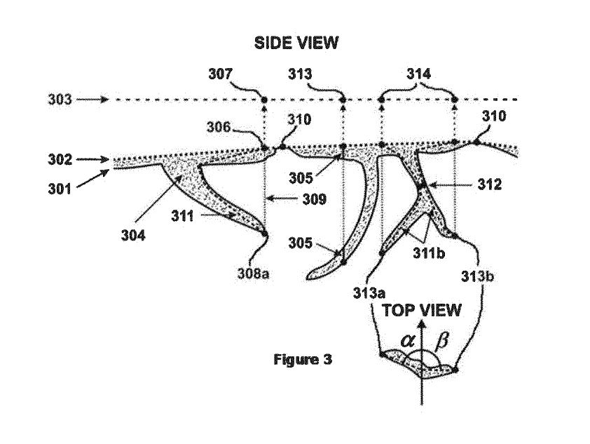

An assessment system according to the disclosure for determining at least one of macro-topology, milli-topology, micro-topology and nano-topology of at least one interface of at least two media includes means 104 for obtaining information on the topology of the of at least one interface of at least two media. The system includes a processing unit 106 for processing the obtained information on the topology of the of at least one interface of at least two media. Exemplary main steps for processing, for example, 3D data as the obtained information to describe the TSAC (Top Surface of Articular Cartilage) are boxed with a dashed line in FIG. 1:
1. A sample volume is segmented from the background using methods known to the art such as (i) volumetric filtering (e.g. Mean, Median, Gaussian or Wiener filter), the preferred method being Gaussian filtering [The Gaussian filter parameters can range: kernel size 3×3×3 to 11×11×11, preferred 5×5×5; sigma 0.65 to 5 (voxels), preferred 1.2.]), (ii) segmentation (e.g. thresholding [global or seeded region growing] by K-means or C-means, exemplary preferred method C-means [The C-Means parameters range from: exponent 1-5, preferred 2.2; probability change converge limit 0.1-0.000001, preferred 0.0003]; the optimal values for the background and segmented volume are found iteratively; for the background the initial guess is the minimum value whereas for the ROI the initial guess is maximum value; minimum sample probability 0.1-1, preferred value 0.6]), (iii) post-filtering, and (iv) speckle removal (Post segmentation filtering and speckle removal can be done using volumetric median filtering, and region-growing-based volume flipping, preferred volume flipping; the parameters for volume flipping range from 0-0.3×volume voxel count, preferred value 0.05×volume voxel count).
2. A simple reference surface is generated, e.g., using iterative surface generation and Delaunay triangularization to local maxima. In more detail, a simple reference surface is generated using iterative surface generation and Delaunay triangularization to local maxima: first is generated an unambiguous sample surface by finding the first “sample” voxel coordinate when approaching from the outside surface nearly orthogonally towards the sample surface. The reference surface is iteratively calculated by first selecting seed points from the edge of the arbitrarily positioned ROI area, then calculating triangle vertexes to these seed points, and then fitting a surface to calculated vertexes, and then calculating the difference between the unambiguous surface and trianglewise fitted surface. Each triangle point with the highest angle is added to the seed point list. This process is then repeated until no new points are found.
3. Voids are extracted (e.g., simple region grow approach on the volume between the reference surface and TSAC) and are analyzed to provide the complex multivalued surface shape information. This analysis is carried out by determining the volume (i.e., the void generated by, e.g., macro/milli/micro/nano-scale fibrillation and fissures in AC) between the reference surface and sample surface using methods known to the art. In more detail, the voids are extracted and analyzed to provide the complex surface shape. This analysis is done by determining the volume between the reference surface and sample surface using region growing. In practice one applies a region growing algorithm to the segmented volume which is limited by the piecewise fitted reference surface, the selected volume of interest, and the sample voxels (FIG. 2).
4. Quantitative mapping (e.g. the tortuosity-like measure defined in FIG. 3) of the voids is locally determined with high spatial resolution. These clefts are pathologically important as they are known to potentially develop into complex fissures that are clinically relevant for disease staging and prognosis.

FIG. 2 demonstrates an example of how the reference surface and the void are identified from TSAC that has been segmented as previously described. For simplicity, a 2D presentation is used to demonstrate the principle of the procedure applied in 3D:
Step 1: starting point representing the segmented TSAC.
Step 2: The data points representing extreme boundaries of the TSAC are identified (black dots).
Step 3: A simple reference surface connecting the data points within extreme boundaries is generated.
Step 4: Local maxima (upper two black dots) of simple reference surface are identified.
Step 5: The local maxima are included into the new simple reference surface and the previous simple reference is discarded.
Steps 4 and 5 are repeated until the simple reference surface is no longer spatially modified or until the spatial modification for each iteration becomes negligible.
Step 6: The void between the simple reference surface (also referred to by reference surface) and the TSAC are identified by, e.g., simple region-growing.

Alternative approaches to determine the reference surface are, e.g., (i) known or arbitrary low-pass filtering of the height information on the TSAC map or (ii) fitting a function to the points representing the TSAC (e.g., spline, bilinear, bicubic, and/or any polynomial).

Examples of biomarkers that can be quantitatively mapped at high spatial resolution are briefly described in the following:
1. Max depth of the voids is a biomarker that can be quantitatively mapped. Void depth is the shortest distance between a point on the reference surface and the most distant point on TSAC beneath the reference point.
2. Tortuosity-like parameter describes the tortuosity of voids. The tortuosity-like parameter is calculated by finding the shortest route from the bottom of the void beneath a reference point to a reference point on the reference surface and by normalizing this by the max void depth beneath the reference point.
3. Depth-wise integral describes the quantity of void voxels beneath a point within the reference surface.
4. Complex void volume is calculated as the sum of the void voxels “trapped” between the TSAC and reference surface.
5. Simple void volume is calculated as the sum of the void voxels “trapped” between the TSAC and reference surface, when the ambiguous (multivalue) TSAC is mathematically simplified to an unambiguous TSAC.
6. The ratio of Complex void volume and Simple void volume is also a biomarker that can be quantitatively mapped.
7. Local thickness is a spatially varying variable, which describes the diameter of the largest sphere that can be fitted into the void. All voxels within this sphere will acquire the value of the sphere diameter. Thus, every voxel within the void will have a value >0. All local thickness values within the volume are eventually converted to a local thickness histogram.
8. The surface ratio is calculated as the ratio of total TSAC area and reference surface area.

FIG. 3 shows a graphical presentation of the quantitative characterization of the complex top surface of AC. 301 represents the TSAC, 302 is the reference surface and 303 is quantitative map to which the parameter values, e.g., maximum depth of the voids, tortuosity-like parameter or depth-wise integral, are recorded. The reference surface 301 in this exemplary embodiment goes through local maxima 310 or the TSAC 302. In the following, the exemplary quantitative maps are described.

Maximum depth of the voids is an exemplary quantitative map, in which the volume “trapped” or enclosed between 301 and 302 is the void 304. Point 308a represents the deepest point of TSAC 301 beneath a reference point 306 on the reference surface 302. The distance 309 representing the recorded maximum depth is presented in the quantitative map (point 307), when maximum depth map is generated.

Tortuosity-like parameter map 311 represents the shortest route 311 from a point 308a on TSAC 301 to reference point 306. The tortuosity-like parameter is defined as the ratio of distance 311 and distance 309 and is recorded and presented as point 307, when a tortuosity-like parameter map is generated.

Depth-wise integral is also an exemplary quantitative map, in which Count of voxels 305, beneath a point belonging to reference surface 302 are recorded and presented as point 313 on the quantitative map 303.

Complex fissure form, i.e. splitting of fissures, can be an important parameter addressing the stage of OA. The splitting of fissures can be identified, e.g., as follows: The extremities 313a, 313b of fissures on TSAC 301 are first identified beneath points on the reference surface 302. The shortest paths 311b from these extremities to points on reference surface are then identified. When these paths are closer to each other than a criterion distance 312, the orientation of the path is determined from the projection to reference surface 302. If the orientation angles are different, the paths are recognized as originating from different extremities, permitting identification of existing or non-existing presence of fissure splitting.

According to an exemplary embodiment, 3D data obtained by a micro-CT machine imaging excised human AC is analyzed. The proposed method is robust enough to work with data generated by different imaging settings (acceleration voltage, current, acquisition time, aperture, number of projections, beam filtering). This means that the need for machine calibration is decreased. This approach can provide considerable advantages. Unlike existing methods to characterize AC as an objective, it is not restricted to 2D, nor does it provide merely global bulk measures, nor does it provide measures that are artificial in the sense that they are not derived from pathological knowledge, nor is it restricted to unambiguous simple surfaces. Thus, issues related to slow subjective assessment without unknown confidence limits are mostly avoided. In addition, the approach is suitable for images obtained in vitro or in vivo. It, therefore, opens up a possibility for 1. to be applied in international classification standards and 2. to be used the approach in education of physicians and medical engineers, and 3. to be used in research, clinical work and drug development. In summary, the above advantages mean that the present method and computer program provide significant improvements for pathological evaluation, diagnosing and therapy of OA compared to existing methods.

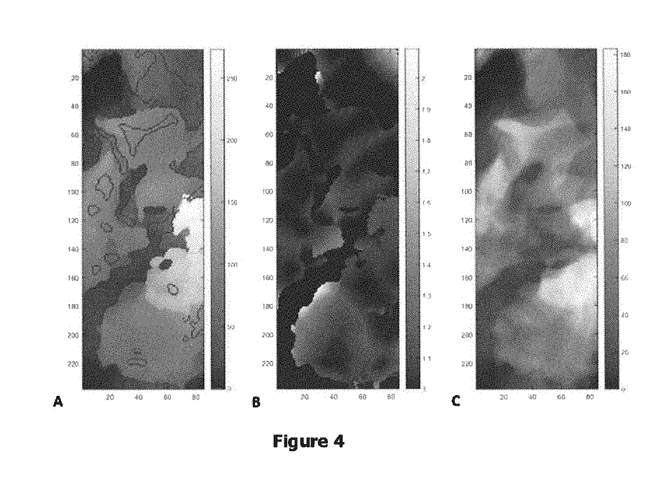

FIGS. 4A-C present exemplary quantitative maps of Maximum depth of voids (A), Tortuosity-like parameter (B) and Depth-wise integral in osteoarthritic AC. The AC samples were obtained by consenting volunteers under existing IRB protocols. The excision and sample preparation is described in Nieminen et al 2015 (Osteoarthritis Cartilage. 2015; 23(9):1613-21). These images were obtained by μCT (80 kV, 150 μA, 1600 projections, 750 ms acquisition time, 5× averaging) and reconstruction was done using the commercial software provided by the instrument manufacturer. The resolution in x, y, and z is 3.0 μm. High contrast areas in FIG. 4A represent a high value and low contrast areas represent a low value. The dark contours in FIG. 4A represent exemplary edges between unambiguous and ambiguos TSAC areas.

In the following, preferred exemplary embodiments are presented by referring to FIGS. 1-4. An assessment system according to exemplary embodiments of the disclosure determines at least one of macro-topology, milli-topology, micro-topology and nano-topology of at least one interface of at least two media. The system includes means 104 for obtaining information on the topology of at least one interface of at least two media. The means 104 can be based, e.g., on devices/modules for performing one or more of the following techniques: optical microscopy, ultrasound microscopy, ultrasound imaging, photo-acoustic imaging, fluorescence microscopy, Raman microscopy, microscopic Fourier transform infrared imaging (FTIR), ultraviolet imaging, interferometric microscopy, diffraction, dynamic light scattering, and scanning electron microscopy. The system can include a processing unit 106 for processing the obtained information on the topology of the at least one interface of at least two media by performing segmentation, in which volume information of the obtained information is segmented from background information of the obtained information. The obtained information is further processed by generating reference surface information, and obtaining information on voids. The information on voids is analyzed to provide multivalued surface shape information. Then in the processing quantitative mapping is performed of the information on voids on the basis of the multivalued surface shape information for determining at least one of macro-topology, milli-topology, micro-topology and nano-topology of at least one interface of at least two media. In an exemplary embodiment according to the present disclosure, the system can include a processing unit 106 for processing the obtained information by applying a region growing algorithm to the segmented volume information which is limited by the piecewise fitted reference surface, the selected volume of interest, and the sample voxels. The processing unit 106 can be any kind of computer or equivalent including at least one processor in which implementation of the embodiments according to the present disclosure can be performed by at least computer program and/or needed algorithms.

In an exemplary embodiment the system can include the processing unit 106 for processing the obtained information on the topology of the of at least one interface of at least two media by extracting voids on the basis of the segmented volume information and reference surface information. The obtained information can be processed by using parameters which are dependent on depth of voids. In a further exemplary embodiment the parameter values can be based on splitting of fissures.

It is also possible to process the obtained information in the processing unit 116 by determining roughness topology of the multivalued surface of the at least one interface on the basis of a mathematical equation which enables determination of more than one value z-value for every coordinate x and y on the interface in Cartesian coordinates.

In a preferred exemplary embodiment according to the present disclosure the assessment system is a medical assessment system. The interface of at least two media can be, e.g., an ambiguous top surface of articular cartilage (TSAC) 301. The system can include a processing unit 106 for processing the obtained information on the topology of the top surface of tissue by performing quantitative mapping in which is recorded at least one of parameter values such as maximum depth of the voids, a tortuosity-like parameter and depth-wise integral to define topology. The obtained information can also be processed by determining at least one parameter map in order to obtain information on tissue failures. In an exemplary embodiment, key features of degenerative grades of OA are defined on the basis of quantitative mapping.

According to an exemplary embodiment, the quantitative maps are used to define key features of the degenerative grades as defined by a grading system relying on AC surface topology, e.g. Pritzker et al. (Osteoarthritis Cartilage. 2006 January; 14(1):13-29; i.e. OARSI grading) of AC as detailed in the following. Clinically relevant grades are 0-3, since a less progressed OA (grades 1-3) would have a better prognosis during therapy as compared more advanced OA (grades 4-6). In the following we discuss image parameters that can be used to identify grades 0, 1, 2, and 3 in the OARSI grading described by Pritzker et al. Intact surface, according to Pritzker et al 2006, can be identified from one of the quantitative maps, e.g. as a small mean or maximum value of maximum depth (e.g. <15 μm). This can be used in identifying grades 0 and 1 as an indicator of surface intactness. Fibrillation through superficial AC layer can be identified as more extensive roughness, e.g. as greater mean of maximum depth (e.g. >15 μm and <200 μm). This can be used as a mean feature to identify grade 2. Vertical fissures can be identified e.g. from values of a maximum depth map (e.g. values >200 μm).

According to an exemplary embodiment, the roughness topology of a multivalue surface of AC or other material can be determined using a mathematical equation. E.g., for an unambiguous surface (simple surface) in 3D (contains x-, y- and z-axes), there can be only one coordinate (x, y) for every z-value on an interface in Cartesian coordinates. When TSAC is considered, which is, for example, a multivalued surface, on a multivalued surface (ambiguous surface), for every coordinate (x, y) on TSAC there can be more than one z-coordinate. A standard roughness parameter, root-mean-square (RMS) roughness, can be determined for an unambiguous surfaces (simple surfaces) as follows:

$${R}_{q} ( x , y ) = \sqrt{\frac{1}{n} {\sum }_{i = 1}^{n} {( \overline{z} - {z}_{i} ( x , y ) )}^{2}} ,$$

where z̅ is a mean value of the surface (see e.g. ISO 25178-2:2012). However, a multivalued surface would be ambiguous; thus, the current standard formulation cannot be applied, because they are only defined for ambiguous surfaces. On a multivalued surface, every point on the TSAC would be a function of (x, y, k(x, y)), k∈ [symbol] , where k is the number of interface z coordinates mapped at (x, y). Thus, one way to determine the root-mean-square roughness for a multivalued surface would be

$${R}_{q , c} ( x , y , z ) = \sqrt{\frac{1}{n} {\sum }_{i = 1}^{n} {( \overline{z} - {z}_{i} ( x , y , k ( x , y ) ) )}^{2}} ,$$

where subscript c stands for ‘complex’ and subscript i represents the index of a point on TSAC. The strength of this formulation is that it takes into account the complexity of a multivalued surface, when the characterized surface is a multivalued surface; however, it provides a standard RMS roughness, if the surface is an unambiguous surface. The roughness parameter could be calculated based on any known function whose parameters are (x, y, k(x, y)). Examples are expansions of standard equations.

According to an exemplary embodiment according to the present disclosure, objective and clinically relevant AC top surface, bone cartilage interface, and tidemark characterization can be achieved by analyzing 3D imaging data similarly to what is described above related to the other embodiments according to the present disclosure. The characterization can be fully automatic. The imaging can be carried out by any suitable means 104 capable of obtaining information about the structure of AC. Examples include optical microscopy, ultrasound microscopy, ultrasound imaging, photo-acoustic imaging, fluorescence microscopy, Raman microscopy, microscopic Fourier transform infrared imaging (FTIR), ultraviolet imaging, interferometric microscopy, diffraction, dynamic light scattering, and scanning electron microscopy. Possible methods are also contacting methods like AFM. The imaging techniques as such are known per se and can be directed to small volumes as required by the embodiment to obtain information about the cartilage sample. Suitable imaging devices are commercially available or can be commercially available in the future and are customizable for the present needs.

According to a further exemplary embodiment, at least one of confidence limits and probability of correct classification for the extracted quantitative maps are determined automatically or semi-automatically. This information can be linked to clinical or pathological information used for at least one of image-guided therapy, diagnosis, self-diagnosis, tele-medicine (exploiting e.g. cloud drive services), prognosis, follow-up of disease progression or regeneration of tissue during therapy (e.g., localized drug delivery into AC) in at least one of clinical (e.g., hospital) and non-clinical setting (e.g., home or austere setting) in at least one of in vivo or in vitro setting. The sample can be of biological or non-biological origin.

According to an exemplary embodiment, at least one of the extracted features and probability of correct classification are linked to existing OA grades by means of, e.g., a look up table.

According to a further exemplary embodiment, the method and computer program can be used for technical buildup and erosion analysis, for example bottom-up-engineering-like 3D printing and ALD processing, erosion studies (i.e., natural or manmade), for instance lithography, landscape erosion, and asteroid characterization.

According to an exemplary embodiment, computation of the desired characteristic features is carried out while the sample is inside the imaging unit or after the sample has been imaged. The imaging can also be done in an iterative manner; i.e., one first gets a rough estimate that gets more and more precise with time.

As described in this description and the related figures, the material assessment system can include as means for importing the obtained information from the means, 104, e.g. a data import module that can handle the 3D image output of the imaging unit, and a data-analysing unit 106 for receiving the obtained information. The material assessment system according to the present disclosure includes processor based means for performing the desired or necessary method steps such as, e.g.: the data-analysing unit having algorithmic means for processing the obtained information on the topology of the of at least one interface of at least two media by performing segmentation, in which volume information of the obtained information is segmented from background information of the obtained information, means for generating reference surface information, means for obtaining information on voids, means for analyzing the information on voids to provide multivalued surface shape information, and means for performing quantitative mapping of the information on voids on the basis of the multivalued surface shape information.

The detailed description of the reference surface generation is an exemplary embodiment, and the reference surface generation can also be performed by other kind of methods. The reference surface can be any surface described by any function and numerically fitted or manually positioned to a location near the sample surface. The reference surface can be located above or below the TSAC or partially crossing the TSAC.

Although the invention has been presented in reference to the attached figures and specification, the invention is by no means limited to those, as the invention is subject to variations.

Thus, it will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. A material assessment system for determining at least one of macro-topology, milli-topology, micro-topology and nano-topology of at least one interface of at least two media, the system comprising:
means for obtaining information on a topology of at least one interface of at least two media;
means for importing the obtained information from the obtaining means;
a data-analysing unit for receiving the obtained information, the data-analysing unit having algorithmic means for processing the obtained information on the topology of the at least one interface of at least two media by performing segmentation, in which volume information of the obtained information is segmented from background information of the obtained information;
means for generating reference surface information;
means for obtaining information on voids;
means for analyzing the information on voids by applying a region growing algorithm to provide complex multivalued surface shape information;
means for performing quantitative mapping of the information on voids based on the multivalued surface shape information; and
wherein the data-analysis unit is configured for determining at least one of macro-topology, milli-topology, micro-topology and nano-topology of at least one interface of at least two media, said data-analysing unit being configured for processing the obtained information on the topology of the at least one interface of at least two media by determining roughness topology of the multivalued surface of said at least one interface based on a mathematical equation.

2. An assessment system according to claim 1, wherein the assessment system is a medical assessment system.

3. A medical assessment system according to claim 2, wherein an interface of at least two media to be analyzed is an ambiguous top surface of articular cartilage (TSAC).

4. An assessment system according to claim 1, comprising:
a processing unit of the data analysing unit for processing the obtained information on the topology of the at least one interface of at least two media by extracting voids based on the segmented volume information and reference surface information.

5. An assessment system according to claim 4, comprising:
a processing unit of the data analysing unit for processing the obtained information by using parameters which are dependent on depth of voids.

6. An assessment system according to claim 5, comprising:
a processing unit of the data analysing unit for processing the obtained information on the topology of at least one interface of at least two media by determining parameter values based on splitting of fissures.

7. A medical assessment system according to claim 2, comprising:
a processing unit of the data analysing unit for processing the obtained information on the topology of the top surface of tissue by performing quantitative mapping by recording parameter values which include at least one of maximum depth of the voids, a tortuosity-like parameter and a depth-wise integral to define topology.

8. An assessment system according to claim 1, comprising:
a processing unit of the data analysing unit for processing the obtained information on the topology of the of at least one interface of at least two media by applying a region growing algorithm to the segmented volume information which is limited by a piecewise fitted reference surface, a selected volume of interest, and sample voxels.

9. A medical assessment system according to claim 2, comprising:
a processing unit of the data analysing unit for processing the obtained information on the topology of at least one interface of at least two media by determining at least one parameter map in order to obtain information on tissue failures.

10. A medical assessment system according to claim 2, comprising:
a processing unit of the data analysing unit for defining key features of degenerative grades of OA (osteoarthritis) based on quantitative mapping.

11. An assessment system according to claim 1, comprising:
a processing unit of the data analysing unit for processing the obtained information on the topology of the of at least one interface of at least two media by determining roughness topology of the multivalued surface of said at least one interface based on a mathematical equation, which enables using at least one said interface on which every x and y coordinate on the interface in Cartesian coordinates has more than one z-value for characterizing the topology.

12. A material assessment method for determining at least one of macro-topology, milli-topology, micro-topology and nano-topology of at least one interface of at least two media, wherein the method comprises:
obtaining information on the topology of at least one interface of at least two media;
importing the obtained information to data-analysis, wherein the obtained information on the topology of the at least one interface of at least two media is processed by performing segmentation, in which volume information of the obtained information is segmented from background information of the obtained information;
generating reference surface information and obtaining information on voids;
analyzing the information on voids by applying a region growing algorithm to provide complex multivalued surface shape information;
quantitatively mapping the information on voids based on the multivalued surface shape information; and
determining at least one of macro-topology, milli-topology, micro-topology and nano-topology of at least one interface of at least two media, by processing the obtained information on the topology of the at least one interface of at least two media by determining roughness topology of the multivalued surface of said at least one interface based on a mathematical equation.

13. An assessment method according to claim 12, wherein the assessment method is medical assessment method.

14. A medical assessment method according to claim 13, wherein the interface of at least two media is an ambiguous top surface of articular cartilage (TSAC).

15. An assessment method according to claim 12, comprising:
processing the obtained information on the topology of the at least one interface of at least two media by extracting voids based on the segmented volume information and reference surface information.

16. An assessment method according to claim 15, comprising:
processing the obtained information by using parameters which are dependent on depth of voids.

17. An assessment method according to claim 12, comprising:
processing the obtained information on the topology of at least one interface of at least two media by determining parameter values based on splitting of fissures.

18. A medical assessment method according to claim 13, comprising:
processing the obtained information on the topology of the top surface of tissue by performing quantitative mapping in which is recorded a parameter value including at least one of maximum depth of the voids, a tortuosity-like parameter and a depth-wise integral.

19. An assessment method according to claim 12, comprising:
processing the obtained information on the topology of the at least one interface of at least two media by applying a region growing algorithm to the segmented volume information which is limited by a piecewise fitted reference surface, a selected volume of interest, and sample voxels.

20. A medical assessment method according to claim 12, comprising:
processing the obtained information on the topology of at least one interface of at least two media by determining at least one parameter map in order to obtain information on tissue failures.

21. A medical assessment method according to claim 13, comprising:
defining features of degenerative grades of OA (osteoarthritis) based on quantitative mapping.

22. An assessment method according to claim 12, comprising:
processing the obtained information on the topology of at least one interface of at least two media by determining roughness topology of the multivalued surface of said at least one interface based on a mathematical equation, which enables using at least one said interface on which every x and y coordinate on the interface in Cartesian coordinates has more than one z-value for characterizing the topology.
